(19) United States
(12) Patent Application Publication
(10) Pub. No.: US 2005/0037375 A1
(43) Pub. Date: Feb. 17, 2005

(54) Screening method for ligand-protein interactions

(76) Inventors: Vimal D. Mehta, Guilford, CT (US); Thomas P. Jarvie, Branford, CT (US); Jonathan M. Rothberg, Guilford, CT (US)

Correspondence Address: Jenell Lawson;Intellectual Property, CuraGen Corporation, 555 Long Wharf Drive, New Haven, CT, 06511, US

(21) Appl. No.: 10/748,805

(22) Filed: Dec. 29, 2003

Related U.S. Application Data
(63) Continuation-in-part of application No. 09/351,617, filed on Jul. 12, 1999, now abandoned.
(60) Provisional application No. 60/094,450, filed on Jul. 28, 1998.

(30) Foreign Application Priority Data
Jul. 28, 1999 (WO) PCT/US99/17055

(51) Int. Cl. C12Q 1/68; G01N 33/53; G01N 33/567

(52) U.S. Cl. 435/6; 435/7.2

(57) ABSTRACT

A rapid method and kit is provided for identifying the targets of biologically active small molecules to identify new drugs that are capable of specific therapeutic effects as well as to identify novel small molecules including agonists and antagonists that may bind selected targets.

This is a Continuation-in-part of prior-filed United States patent application U.S. Ser. No. 09/351,617, filed July 12^{th} 1999, which in turn claims priority of provisional patent application U.S. Ser. No. 60/094,450, filed July 28^{th} 1998.

### FIELD OF THE INVENTION

The present invention relates to a method for screening small molecules (such as pharmacological agents) that bind to selected cellular targets, for those targets capable of binding selected small molecules, and a kit containing the reagents for conducting the method.

### BACKGROUND OF THE INVENTION

A basic area of inquiry in pharmacology and medicine is the determination of ligand-receptor interactions. The pharmacological basis of drug action, at the cellular level, is quite often the consequence of non-covalent interactions between therapeutically relevant small organic molecules and high affinity binding proteins within a specific cell type. These small organic ligands may function as agonists or antagonists of key regulatory events that orchestrate both normal and abnormal cellular functions. For years the pharmaceutical industry's approach to discovering such ligands has been based upon the random screening of thousands of small molecules in specific in vitro and in vivo assays to determine potent lead compounds that may then be used to further industry's drug discovery efforts. In many instances, such lead compounds often exert very well-defined effects with regard to cell function, as, for example, inhibition of cytokine production, DNA replication, or the like. However, the mechanism of action at the molecular (that is the ligand-protein interaction) level has remained elusive. There is thus an unmet need for a general, efficient and sensitive method to identify the cellular targets for such pharmacological agents in order to accelerate the search for novel drugs both at the basic and applied levels of research.

At the time of making the present invention, no efficient methodology existed for rapidly identifying a biological target as, for example, a protein for a particular small molecule ligand. Prior and existing approaches to detect and isolate putative target proteins include the use of affinity chromatography, radio-labeled ligand binding, and photoaffinity labeling taken in combination with protein purification methods. These approaches were followed by the cloning of the specific gene encoding the appropriate target protein based on the peptide sequence of the isolated target. Each of these approaches depend upon the abundance of the putative target protein in the sample; and unfortunately, each of these approaches are time consuming and labor intensive. Thus, at the time of making the present invention, there was no existing technology allowing for the direct identification of the cDNA encoding a target for a given ligand.

Similarly, at the time of making the present invention, no efficient general approach existed for identifying a small molecule capable of binding to any selected cell target regardless of its biological function. Prior attempts to address appropriate approaches to this problem [see, for example, U.S. Pat. Nos. 5,789,184, 5,876,951, 6,100,042, and 6,255,059, the disclosures of which are incorporated in toto herein by reference] have described alternate screening assays directed to identifying molecules capable of binding cell surface receptors so as to activate a selected signal transduction pathway. More accurately, these four cited United States patents describe modifications within selected yeast signaling pathways so as to mimic steps in the mammalian signaling pathway. However, each of their approaches was specific for certain signaling pathways and thus has limited utility for broadly discovering small molecules that interact with any cellular target as is capable and in accordance with the present invention.

Recently, a yeast genetic screening method has been reported in the literature for specifically identifying protein-protein interactions in an in vivo system. This screening method is described in U.S. Pat. Nos. 5,468,614, 5,469,285, and in Nucleic Acid Research 23:1152, 1995, the disclosures for each of which are incorporated in toto herein by reference. The genetic screening method described in these documents relies upon the interaction of two fusion proteins to bring about the transcriptional activation of a reporter gene such as E. coli derived β-galactosidase (Lac Z). One of the two fusion proteins used in this method comprises a pre-selected protein fused to the DNA binding domain of a known transcription factor, while the second fusion protein comprises a polypeptide from a cDNA library fused to a transcriptional activation domain. In order for the reporter gene to be activated, the polypeptide from the cDNA library must bind directly to the pre-selected target protein. After binding, yeast cells harboring an activated reporter gene may then be differentiated from other cells within the population, and the cDNA encoding for the interacting polypeptides can subsequently be isolated and sequenced. The application of the yeast genetic method described in these documents has also been reported to have been adapted to screening of peptide combinatorial libraries and protein interactions [see Nucl. Acids Res. 23:1152 (1995)]. However, the screening methodology described in these documents is not suitable for screening small molecule-protein interactions because it relies solely on genetically encoded fusion proteins.

There is thus an unmet need for a general screening method to determine the interaction of small molecules and protein targets so as to identify new drugs that are capable of specific therapeutic effects in a variety of disease states, as well as a method to identify agonists and antagonists that may interfere or compete with the binding of the small molecules for these targets.

Recently, a yeast genetic screening method has been reported in the literature [see Proc. Natl. Acad. Sci., 93:12817 (1996)] for detecting ligand-receptor interactions in vivo (hereafter referred to as the “updated assay”). This updated assay was developed as an improvement over the yeast genetic screening method. The feasibility of the updated assay for which a third synthetic hybrid ligand is combined with the prior two-hybrid system was demonstrated using as the hybrid ligand a heterodimer of covalently linked dexamethasone and the immunosuppressive drug FK506. As described, yeast-expressing fusion proteins of the hormone binding domain of the rat glucocorticoid receptor (“GCR”) were fused to a site specific DNA binding domain bound to the promoter of a reporter gene when plated on medium containing the dexamethasone-FK506. Activation of the reporter gene was observed when a second hybrid protein expressing a fusion protein of a transcription activation domain was fused to the FK binding protein FKBP-12 receptor and then dimerized with the first fusion protein via a dexamethasone-FK506 bridge. Using this methodology, a Jurkat cDNA library was screened and overlapping clones of human FKBP12 isolated. A number of factors such as inherent sensitivity and permeability affect the general utility of this updated genetic screening method. For example, affinities in the nanomolar or subnanomolar range (i.e., having a 0.5 nM kd) for both ligand-receptor pairs (e.g., mutant GCR-dexamethasone and FKBP12-FK506) are required, based on the observation that a wild-type hormone binding domain of GCR with a kd of 5 nM could not produce any detectable signal.

Accordingly, it is readily apparent to those involved with such needs that there is still an unmet requirement for improved sensitivity of a general screening method to allow screening of a wide range of ligands and proteins, including but not limited to, ligands derived from combinatorial chemistry libraries and proteins encoded by cDNAs. The present invention addresses this unmet requirement.

### SUMMARY OF THE INVENTION

The invention disclosed herein provides a rapid method and kit for identifying the targets of biologically active small molecules so as to identify new drugs that are capable of specific therapeutic effects as well as to identify novel small molecules, including agonists and antagonists, that may bind selected targets.

The present invention is directed to a method for providing a genetic system capable of detecting pharmacologically relevant small ligand-protein interactions. Furthermore, the method according to the present invention may be used to screen a multitude of proteins for interactions with any small ligand. The intention of this method is to identify the biologically relevant receptor for a pharmacological agent. A further purpose of the present invention is to provide a method for high throughput pharmacological screening in both yeast and mammalian cells to identify novel ligands that bind to known cellular targets. In a preferred embodiment, a method is described for identifying a cellular component to which a small molecule is capable of binding, the method having the following steps: (a) providing a dual-linked hybrid ligand molecule consisting essentially of a ligand “A” linked together to a ligand “B”, wherein ligand A has a specificity for a first predetermined target and forms an irreversible (covalent) bond therewith, and wherein ligand B is the small molecule; and (b) introducing the hybrid molecule into a material sample having an environment containing a first expression vector, including DNA encoding the target for ligand A linked to a coding sequence for a first transcriptional module for expression as a first hybrid protein, a second expression vector including a random DNA fragment encoding a polypeptide fused to a second transcriptional module for expression as a second hybrid protein, and a third vector including a reporter gene wherein the expression of the reporter gene is conditioned on the proximity of the first and second hybrid proteins. The hybrid molecule is permitted to bind to the first hybrid protein through ligand A and to the second hybrid protein through ligand B so as to activate the expression of the reporter gene. Those samples expressing the reporter gene are identified and the second hybrid protein is characterized in the identified samples so as to determine the cellular component to which the small molecule is capable of binding.

By “irreversible (covalent) bond” as used herein is meant a strong bond formed between two chemical components (e.g., “A” and “X” of FIG. 3) by a sharing of orbital electrons. This limitation is essential for the present invention.

In another embodiment according to the present invention, there is demonstrated a method is provided for identifying a small molecule capable of binding a molecular target, comprising the steps of: (a) providing a preparation of a library of hybrid molecules wherein each hybrid consists essentially of two ligands identified as ligand A and ligand B that are linked together, wherein ligand A has a specificity for a first predetermined target and forms an irreversible (covalent) bond, and ligand B is a random small molecule; and (b) introducing the preparation into a sample having an environment containing a first expression vector, including DNA encoding the target for ligand A, linked to a coding sequence for a first transcriptional module for expression as a first hybrid protein, a second expression vector including DNA encoding a second predetermined target for identifying a putative interacting ligand, linked to a coding sequence for a second transcriptional module for expression as a second hybrid protein, and a third vector including a reporter gene wherein the expression of the reporter gene is conditioned on the proximity of the first and second hybrid protein. The hybrid molecules are permitted to bind to the first and second hybrid proteins so as to activate the expression of the reporter gene. Those samples expressing the reporter gene are identified and ligand B corresponding to the interacting ligand, is characterized so as to determine the small molecule capable of binding to the molecular target.

In another embodiment of the present invention, there is demonstrated a method for identifying a small molecule capable of competitively binding a molecular target, in the presence of a known binding ligand, the method having the following steps; (a) providing hybrid molecules consisting essentially of two ligands identified as ligand A and ligand B that are linked together, wherein ligand A has a specificity for a first predetermined target and forms an irreversible (covalent) bond, and ligand B has a specificity for a second predetermined target; and (b) introducing the hybrid molecules into a sample containing an environment having a first expression vector, including a DNA encoding the first predetermined target, linked to a coding sequence for a first transcriptional module for expression as a first hybrid protein, a second expression vector including DNA encoding the second target linked to a coding sequence for a second transcriptional module for expression as a second hybrid protein, a third vector including a reporter gene wherein the expression of the reporter gene is conditioned on the proximity of the first and second target, and at least one random small molecule identified as ligand B. The hybrid ligand molecules are permitted to bind the first and second target to activate the reporter gene in the presence of ligand B. The samples are identified according to the absence of expression of the reporter gene, and ligand B is characterized so as to determine the identity of the small molecule binding competitively to the molecular target.

In still another embodiment of the present invention, a kit is provided for detecting interactions between pharmacologically relevant small molecules and proteins. The kit according to the present invention has the following elements: (i) a pre-activated ligand A and reagents for forming a hybrid molecule with at least one type of a ligand B; (ii) a first expression vector including DNA encoding the binding protein for Ligand A linked to a coding sequence for a first transcriptional module for expression as a first hybrid protein; (iii) a second expression vector including a random DNA fragment encoding a polypeptide linked to a coding sequence for a second transcriptional module for expression as a second hybrid protein; (iv) a third vector including a reporter gene wherein transcription of the reporter gene is conditioned on the proximity of the first and second target proteins; (v) an environment for transcription and translation of the hybrid proteins and reporter genes; and (vi) a means for detecting the expression of the reporter gene following the formation of a trimeric complex between the hybrid ligand and the hybrid proteins.

Other facets of the present invention include, for example, preparation of wild type and mutant libraries for the respective targets (i.e., genes encoding the proteins) for ligand A in order to be able to modulate the affinity of the newly created analogs (modified aspirin and β-lactams) as appropriately required by the assay.

A more thorough and complete understanding of the general screening method for ligand-protein interactions and the usages that this method may be applied can be obtained by reference to the following figures and examples which are presented by way of illustration and are not intended, nor should they be considered, to limit the scope of the invention described and claimed herein.

### BRIEF DESCRIPTION OF THE DRAWINGS

In the figures,

Fig. A is a diagrammatic representation of the mechanism of the eukaryotic transcriptional activator showing two functionally independent domains, a DNA binding domain and an activation domain, where proximal positioning of the two domains triggers transcription.

Fig. B is a diagrammatic representation of the yeast two-hybrid assay showing the interaction between protein X, DNA binding domain (Gal4 or LexA) fusion protein and Y, activation domain fusion protein, expressed by cDNA, which triggers the expression of the reporter gene (His3, LacZ, Ura3) subsequent to the interaction of the transcriptional activator modules with the Gal4/LexA upstream activation sequences.

Fig. C is a diagrammatic representation of the components of the three-hybrid assay showing a known target protein (X), DNA binding domain (Gal4 or LexA) fusion protein, and Y, activation domain fusion protein, expressed by cDNA, and the hybrid ligand A-B that interacts reversibly with the two fusion proteins X and Y resulting in the activation of the reporter genes (His3, LacZ, Ura3) subsequent to the interaction of the transcriptional activator modules with the Gal4/LexA upstream activating sequences.

Fig. D is a diagrammatic representation of the components of the three-hybrid assay showing a known target protein (X), DNA binding domain (Gal4 or LexA) fusion protein, and Y, activation domain fusion protein, expressed by cDNA, and the hybrid ligand A-B that interacts with the two fusion proteins X (A interacts irreversibly to X) and Y (reversible interaction) resulting in the activation of the reporter genes (His3, LacZ, Ura3) subsequent to the interaction of the transcriptional activator modules with the Gal4/LexA upstream activating sequences.

FIG. 1 is a diagrammatic representation of the yeast two-hybrid assay showing the interaction between a fusion protein X, DNA binding domain (Gal4 or LexA) fusion protein and Y, activation domain fusion protein, expressed by cDNA, which triggers the expression of the reporter gene (His3, LacZ, or Ura3) subsequent to the interaction of the transcriptional activator modules with the Gal 4/LexA upstream activating sequences.

FIG. 2 is a diagrammatic representation of the components of the three-hybrid assay showing a known target protein (X), DNA binding domain (Gal4 or LexA) fusion protein, and Y, activation domain fusion protein, expressed by cDNA, and the hybrid ligand A-B that interacts with the two fusion proteins X (A interacts irreversibly to X) and Y (reversible interaction) resulting in the activation of the reporter genes ((His 3, LacZ, Ura3) subsequent to the interaction of the transcriptional activator modules with the Gal 4/LexA upstream activating sequences

FIG. 3 illustrates the mechanism for aspirin and its analogs for irreversible (covalent) bonding to cyclooxygenase.

FIG. 4 is a diagrammatic illustration of the synthesis of the coupling of aminoalkylsalicyclates to dexamethasone.

FIG. 5 illustrate the structures of affinity labeling agents (e.g., Penicillins and Cephalosporins/cephamycins) where R and Y can be replaced with small molecules (e.g., dexamethasone and FK506).

FIG. 6 illustrate the examples of synthesis of hybrid molecules using mechanism-based inactivators e.g., vigabatrin, eflornithine and fluorouracil.

FIG. 7 illustrate the synthesis of fluorescein-EDT2 from Fluorescein and fluorescein-EDT2 is coupled with dexamethasone (carboxylic group)

FIG. 8 illustrate the enzyme catalyzed covalent bond formation using two small molecules.

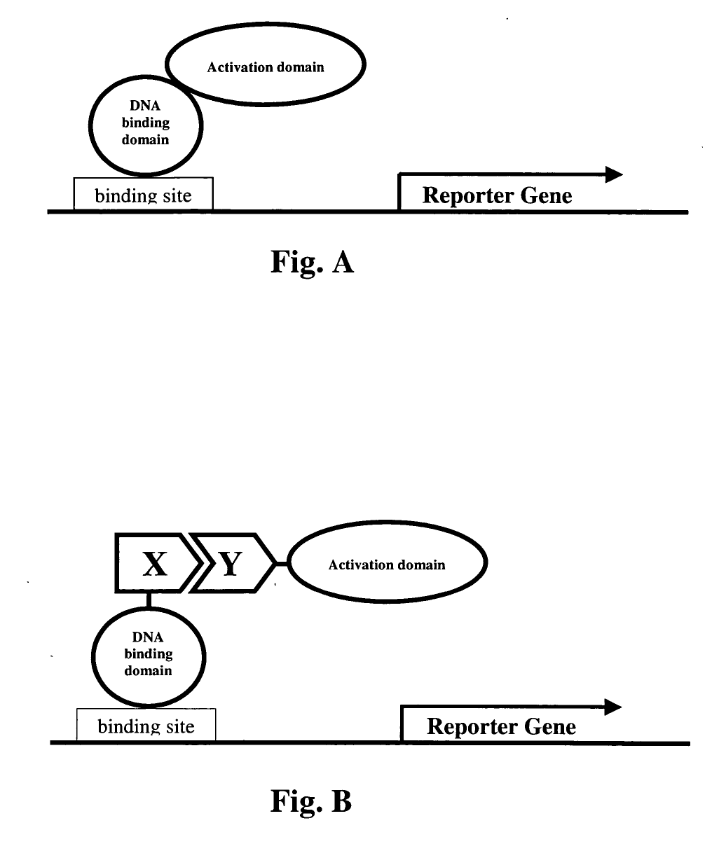

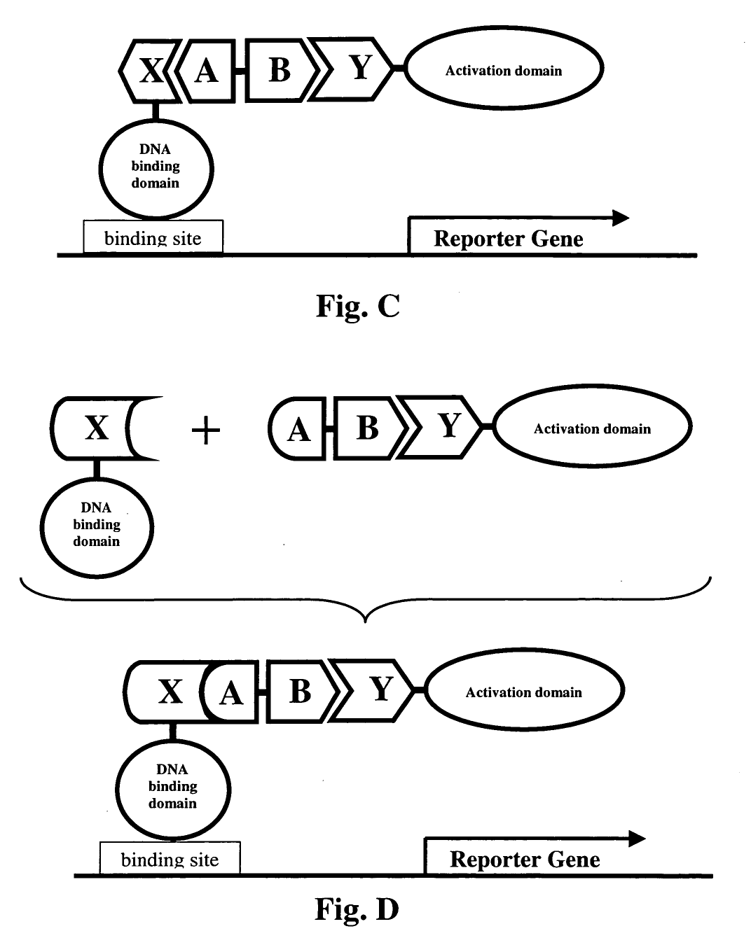

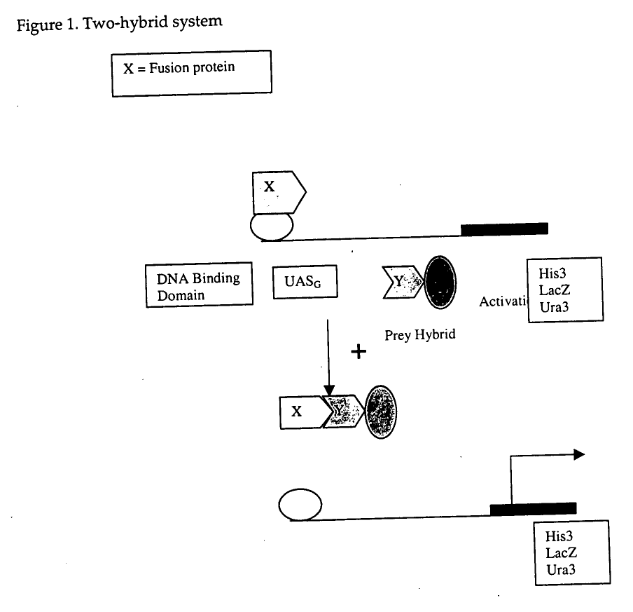

### DETAILED DESCRIPTION OF SPECIFIC EMBODIMENTS

According to the invention as described herein, “a screening assay” is meant to describe a process for selecting or eliminating items by means of at least one distinctive criteria. The screening assay is intended to be distinct from any assay of biological function or effect. The items in this method are small molecules, and the selection is based on capability of binding a target molecule (sometimes called a receptor). A feature of the screening assay is the ability to rapidly examine the binding of large numbers of different small molecules for selected target molecules and conversely, to examine the binding of selected molecules for a large number of target molecules. The positive interaction between small molecules and a target results in a chemical signal that is quantitatively and/or qualitatively different from a signal if any produced in the negative control.

“The sample containing an environment” is defined herein as a sample containing a complex biochemical mixture such as is found within a eukaryotic or prokaryotic cell or alternatively may be formed from a cell lysate maintained in a synthetic boundary such as a membrane or a reaction vessel.

“A cell component” is defined herein as including any of a nucleic acid, a polysaccharide, a lipid, or a protein or any combination of these.

A “reporter gene” is defined herein as a marker for detecting the formation of a hybrid complex. The reporter is not intended in itself to have a therapeutic effect in the environment within which it is located in the assay.

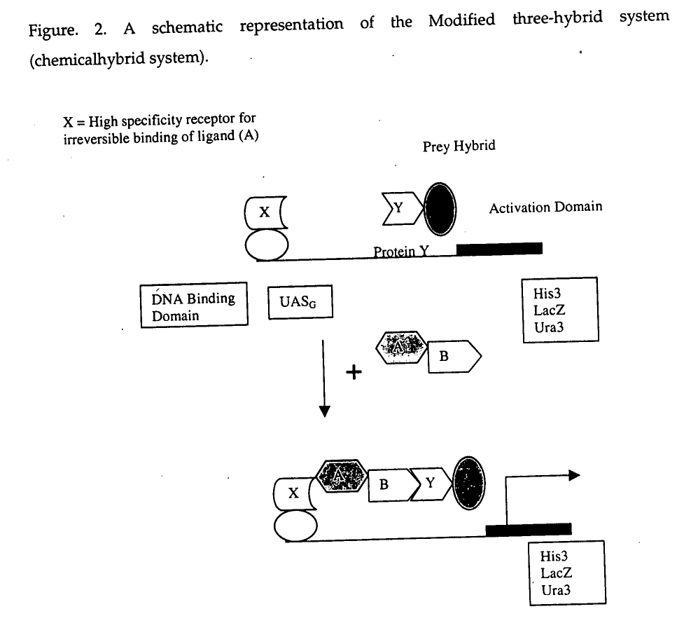

The novel chemical hybrid assay is depicted in FIG. 2 and involves the formation of a complex between a hybrid ligand, and two hybrid proteins in which one component of the chemical hybrid complex may be unknown. The unknown component in the assay may be either the small molecule contained in the hybrid ligand, or one of the hybrid proteins (or both small molecule and protein). There is no requirement that the unknown component be purified prior to the screening assay. Indeed, it is expected that the unknown component be contained in a mixture containing a large number of components, some or all being unidentified. These interactions may be determined in vivo or in vitro when the chemical hybrid complex triggers the expression of at least one reporter gene that can be detected by an appropriate assay.

Examples of the utility of the assay include: (1) determining the identity of target molecules having a binding affinity with a known small molecule where the small molecule has pharmacologic activity and where the target molecules may be suited for therapeutic intervention in a variety of disease states; (2) determining the identity of a small molecule capable of direct binding to a known target molecule where the identified small molecules may be suitable as therapeutic agents; (3) determining the identity of a small molecule capable of binding competitively to a known target molecule in the presence of a hybrid molecule so as to inhibit the binding between the target and the pre-selected small molecule; (4) developing a high throughput pharmacological assay in a number of cell types and organisms to screen for drug candidates; and (5) selecting novel small molecule for binding novel targets with high affinity using an iterative process of direct and competitive screening steps. For example a known small molecule may be used to identify a target and subsequently the target may be used to identify a novel small molecule. This approach can provide novel small molecule pharmacologic agents and may also provide highly specific reagents for use in screening for small molecules in the environment. The advantages of such an assay are described in, for example, PNAS 93, 12817-12821 (1996).

The method according to the present invention described herein is identified as a chemical-hybrid system that includes the step of providing a hybrid molecule consisting of two ligands, for simplicity identified as ‘ligand A’ and ‘ligand B’ that are linked together, wherein ligand A has a specificity for a first predetermined target and forms an irreversible (covalent) bond, a necessary requirement of the present method; and ligand B is the small molecule (FIG. 2). More specifically, the present invention describes and provide a set of novel hybrid molecules that form an irreversible bond to the predetermined target and hence resulting in a modality herein referred to as a previously unknown chemical-hybrid system. There are several obvious advantages of the new screening system such as enhanced sensitivity, specificity and thus allowing screening of wide range of ligands and proteins (permit detection of both strong and weak ligand-protein interactions).

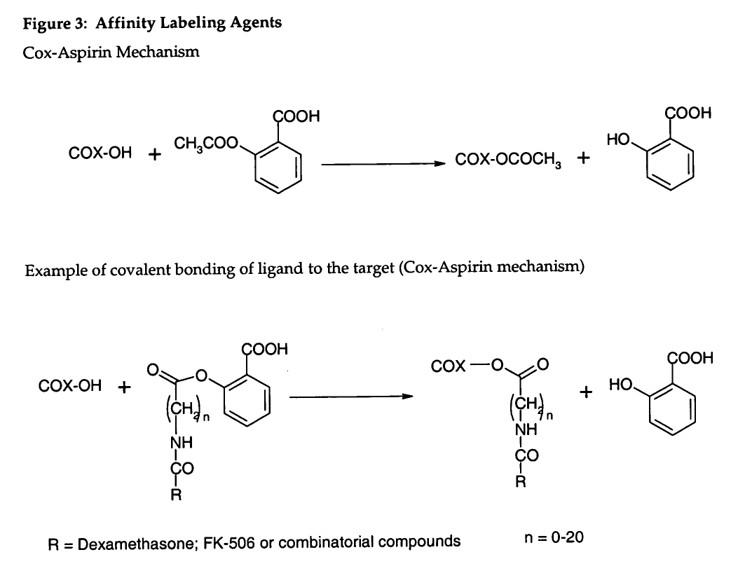

The invention further describes irreversible ligands for the chemical-hybrid assay, including, but not limited to the following options. The test system utilizes small molecule (ligand A) as discussed below and exemplified in Example 1 and FIGS. 3-8.

1. Affinity Labeling Agents (Chemically Reactive Compounds): Synthesis of Hybrid Molecules

An affinity labeling agent is a reactive compound having a structure that is similar to that of the substrate for a target enzyme. Subsequent to reversible complex formation, the affinity labeling agent will generally react with the active site nucleophiles, i.e., amino acid side chains by acylation or alkylation, thereby forming a stable covalent bond to the enzyme. If the molecule has a very low Ki for the target enzyme, then complex formation will be favored, and the selective reactivity will be enhanced. Another approach to increase the selectivity of this class of inactivators is to modulate the reactivity of the active functional group.

The hybrid molecule in this embodiment consist of a small molecule (ligand A) which is derived from a widely recognized drugs (e.g., aspirin and antibiotics β-lactams): penicillins and cephalosporins/cephamycins). These compounds have specificity for cyclooxygenase (Cox-1 and Cox-2) and peptidoglycan transpeptidase respectively to form irreversible (covalent) bond with their targets by acetylation of the amino acid residue, serine hydroxyl group.

(i) Modified Aspirin Analogs:

A part of the present invention also lends itself to describing a method to synthesize hybrid molecules using aspirin-cyclooxygenase irreversible bonding mechanism (synthesis of modified aspirin analogs). Aspirin is a well studied small organic molecule, that is known to bind irreversibly to cyclooxygenase [see J. Biol. Chem. 255:2816 (1980)] with strong binding affinities and to other proteins (e.g., albumin, hemoglobulin, lens crystalline). The aspirin molecule, similar to many proteins, is modular in nature. The modified aspirin molecule irreversibly (covalently) binds with cyclooxygenase (and other proteins) through transesterification; and thus serves as an effective small molecule for proving the efficacy of the chemical-hybrid system.

Modified aspirin (aminoalkyl salicylates) were synthesized as shown in FIG. 3. The dexamethasone (Sigma) and FK506 (Fujisawa Pharmaceuticals) were linked to aminoalkyl salicylates to form a hybrid molecule. The chemistry utilized to effect the linkage is shown in FIG. 3. The dexamethasone and FK506 hybrid molecule with aminosalicylates were synthesized utilizing synthetic transformations outlined in FIG. 3. The dexamethasone portion of the hybrid molecule was synthesized as dexamethasone free amine starting from commercially available dexamethasone in three synthetic modifications [see PNAS 93:12817, (1996)]. The FK506 portion of the hybrid molecule was synthesized as the N-hydroxysuccinamide activated ester from the natural product FK506 in a total of four synthetic modifications. The dexamethasone amine (and FK506 activated ester) were coupled to aminosalicyclates as shown in FIG. 3.

(ii) β-Lactams: Penicillins, Cephalosporins and/Cephamycin

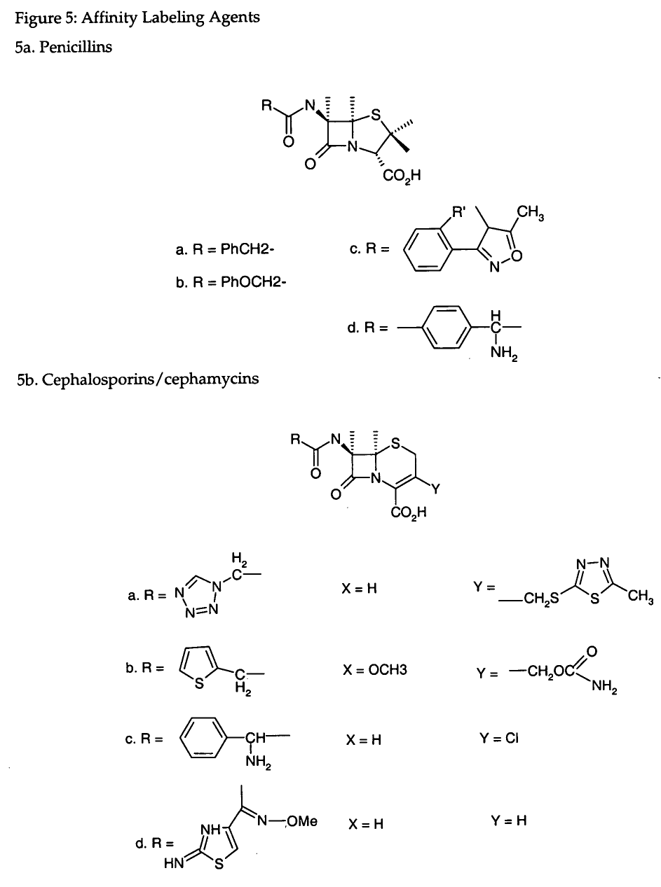

The method of the present invention also provides for a method for creating the hybrid molecule. The hybrid molecule essentially consists of two ligands identified as ligand A and ligand B that are linked together, wherein ligand A has specificity for a first predetermined target and forms the irreversible (covalent) bond that is necessary for the present invention; and ligand B that is the small molecule. It is well known that many antibiotics form an irreversible (covalent) bond with their targets (e.g., peptidoglycan transpeptidase). The penicillins and cephalosporins are ideal drugs in that they inactivate an enzyme that is essential for bacterial growth but does not exist in animals, namely, the peptidoglycan transpeptidase. This enzyme catalyzes the cross-linking of the peptidoglycan to form the bacterial cell wall. The beauty of these antibiotics is that neither are exceedingly reactive; consequently, few nonspecific acylation reactions occur. Their modulated reactivity and nontoxicity make them ideal candidates to be used as irreversible (covalent) inactivators for the chemical-hybrid assay according to the present invention. The modifications in the structure of these antibiotics have been so extensive that essentially every atom excluding the lactam nitrogen has been replaced or modified in the search for improved antibiotics. The designing of the hybrid compounds include replacing R or Y with a small molecule (e.g., dexamethasone and FK-506) using penicillins and cephalosporins listed in Table 1, 1A and FIG. 5 employing standard synthetic manipulations.

2. Mechanism-Based Enzyme Inactivators (Chemically Unreactive Species): Synthesis of Hybrid Molecules

Also describes and encompassed within the present invention is a method to prepare hybrid molecules using mechanism-based enzyme inactivators. A mechanism-based enzyme inactivator is an unreactive compound that bears a structural similarity to the substrate or product for a specific enzyme. Once such a mechanism-based enzyme inactivator binds to the active site, the target enzyme, via its normal catalytic mechanism, will normally convert it to a generally very reactive product, and prior to its escape from the active site, this reactive product, in almost all cases, will form a covalent bond to the enzyme.

The key feature that makes mechanism-based enzyme inactivators suitable to prepare hybrid molecules with irreversible (covalent) bonding characteristics necessary for the present invention is that such inactivators are normally non-reactive compounds. Consequently, nonspecific reactions such as alkylation or acylations with other proteins is not a problem with the method being carried out in accordance with the present invention. In the most ideal case according to the present invention, only the target enzyme will have the capability of catalyzing the appropriate conversion of the inactivator to the activated species.

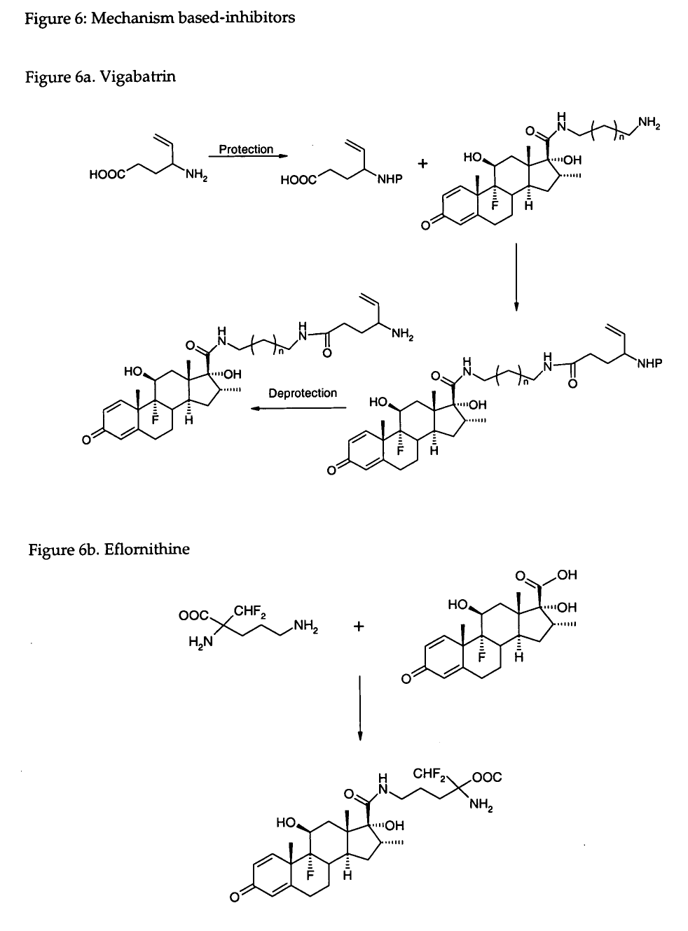

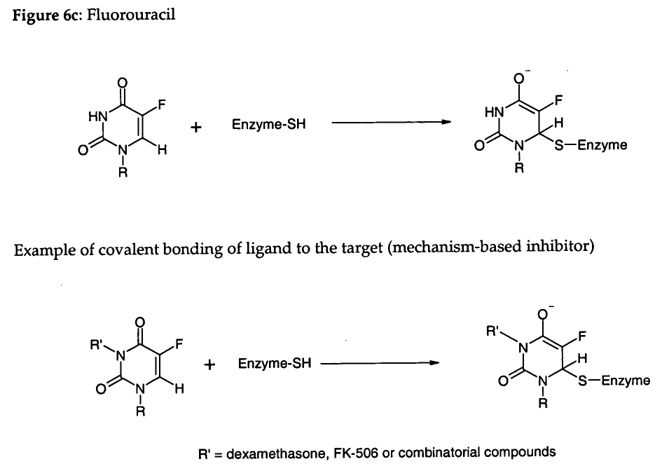

Table 2 provides a list of inactivators for creating hybrid molecules with dexamethasone and FK506. Specific examples of mechanism-based inactivators are outlined in detail in FIG. 6 which will form hybrid molecules with dexamethasone (and FK-506) for testing in chemical-hybrid assay.

furthermore, β-lactamase inhibitors can also act as irreversible ligand A in the invention to synthesize the hybrid molecule. Both clavulanate and sulbactam are potent mechanism-based inactivators of β-lactamase.

3. Covalent Labeling of Recombinant Protein and Engineered Molecules Inside Live Cells: Synthesis of Hybrid Molecules

Another embodiment of the method according to the present invention described herein is that of a method for irreversible labeling of the predetermined target with a hybrid molecule consisting of two ligands identified as ligand A and ligand B that are linked together, wherein ligand A has a specificity for a first predetermined target and forms the necessary irreversible (covalent) bond; and ligand B is the small molecule.

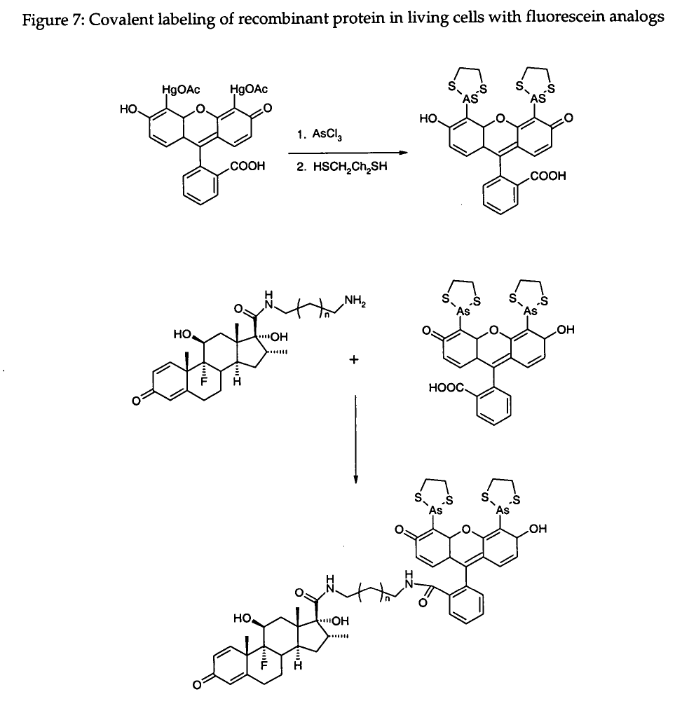

This method comprises engineering a recombinant protein (or small receptor domain) that has a high affinity for a specifically tailored ligand A of the hybrid molecule. Recently, covalent labeling of recombinant protein composed of as few as six natural amino acids in living cells has been reported using the fluorescein analog [see Science 281:269 (1998)]. The approach describes therein exploits the facile and reversible covalent bond formation which occurs between organoarsenicals and pairs of thiols. The hybrid molecule in the invention is synthesized by coupling of the small molecule (e.g., dexamethasone) to fluorescein analog as depicted in FIG. 7. The small receptor domain DNA sequence will be expressed in yeast on binding domain. More such systems can be envisioned and designed by those skilled in the art.

4. Enzyme-Catalysed Covalent Labeling: Synthesis of Hybrid Molecules

In still another embodiment of the present invention, an enzyme-catalysed method is described for covalent labeling of ligand to the target. Recent advances in molecular and structural biology have improved the availability of virtually any biocatalyst in large quantity and have also provided an insight into the detailed functional topology of biocatalysts. These advances increasingly allow the rational exploitation of biocatalysts for use in organic synthesis.

This method involves incubating small molecule having typical functional groups (e.g., NH₂, COOH, SH, OH) in cells wherein an appropriate enzyme (gene encoding; inducible) is used to couple the small molecule to a fusion protein and or peptide (e.g., yeast hybrid system). This is accomplished either directly by having desired functional groups on the small molecule and or on a linker to separate the target from the small molecule in order to minimize any adverse effects of the target on the activity of small molecule.

This method also involves the expression of the specific target protein and/or peptide in the yeast cells (and or a recombinant library of proteins and or peptides). In this embodiment, the target library of protein and/or peptide will be created by the user, using known techniques, with a bias to have desired functionality for efficient covalent bonding (to enhance rate of reaction) with hybrid molecules.

5. Combinatorial Biocatalyses: Synthesis of Hybrid Molecules

In another embodiment of the invention combinatorial biocatalyses method is described for the synthesis of a hybrid molecule essentially consisting of two ligands identified as ligand A and ligand B that are linked together, wherein ligand A has a specificity for a first predetermined target and forms an irreversible (covalent) bond; and ligand B is the small molecule.

Nature's most potent molecules are produced by enzyme-catalysed reactions, coupled with the natural selection of those products that possess optimal biological activity. Combinatorial biocatalysis harnesses the natural diversity of enzymatic reactions for the iterative synthesis of organic libraries. Combinatorial biocatalysis is a powerful addition to the expanding array of combinatorial methods for the generation and optimization of lead compounds in drug discovery and development [see Trends Biotechnol., 16 (5): 210 (1998)]. The methods for the synthesis of the hybrid molecules in the invention may not be limited to the chemical methods. Iterative reactions can be performed using isolated enzymes or whole cells, in natural and unnatural environments, and on substrates in solution or on a solid phase. It includes, but does not limit the scope of the invention, the coupling of ligand A by a biochemical method (enzymatic methods) to the compound (ligand B). See, e.g., Table A. The ligand B may have originated either by the chemical or any biochemical methods (enzymatic biocatalyses) and/or by the combination of both the chemical and enzymatic methods.

TABLE A
Biocatalytic reactions available for combinatorial chemistry

| Reaction Type | Specific Reactions |
|---|---|
| Introduction of | Carbon-carbon bond formation |
| functional groups | Hydroxylation |
| | Halogenation |
| | Halohydrin formation |
| | Cycloadditions |
| | Additions to amines |
| Modification of | Oxidation of alcohols to aldehydes and ketones |
| existing | Reduction of aldehydes and ketones to alcohols |
| functionalities | Oxidation of sulfides to sulfoxides |
| | Oxidation of amino groups to nitro groups |
| | Oxidation of thiols to thioaldehydes |
| | Hydrolysis of nitrites to amides and carboxylic acids |
| | Replacement of amino groups with hydroxyl groups |
| | Lactonization |
| | Isomerization |
| | Epimerization |
| | Dealkylation |
| | Methly transfer |
| Addition onto | Esterification |
| functional groups | Carbonate formation |
| | Glycosylation |
| | Amidation |
| | Phosphorylation |

The present invention also describes still another method for the synthesis of the desired hybrid molecule that utilizes methods found in nature to perform synthesis of compounds (e.g., synthesis of natural products). The hybrid molecule according to the present invention essentially consists of two separate ligands, for ease of reference identified as ligand A and ligand B, that are joined together. In this structural form, ligand A was selected to have a specificity for a first predetermined target and forms an irreversible (covalent) bond with the target that is necessary for all embodiments of the present invention; and ligand B is selected to be the small molecule. According to this specific method being described, the synthesis of this linked molecule is accomplished by the microorganisms where ligand A and B may be inherently present in the microbes or are included (added) in the microbes according to design specifications and requirements, as a viable alternative to those other methods described herein for the synthesis of the desired hybrid molecule.

In contrast to ligand A, ligand B can be a random molecule of unknown identity obtained from a combinatorial library, or other small molecule archive. Examples of combinatorial libraries from which ligand B can be selected include, but are not limited to, peptide libraries, nucleic acid libraries, polysaccharide libraries, and small organic molecules. In addition, libraries produced by combinatorial biocatalyses and collections of environmental molecules and molecules from chemical processes. According to the present invention “small molecule” is as a molecule having a molecular weight of less than 1000 D, more particularly less than 800 D, and greater than 50 D.

The test system exemplified in Example 1, utilizes as ligand A, modified aspirin which irreversibly (covalently) binds to cyclooxygenase; ligand B is FK506 and dexamethasone (which binds to FKBP12 and GCR respectively).

The hybrid linkage between ligand A and ligand B may be formed by any of the methods known in the art [see, for example, Advanced Organic Chemistry (1985) John Wiley & Sons Inc; WO94/18317; WO95/02684; WO96/13613; and WO96/06097].

In one aspect of the present invention, a single ligand or small molecule having electrophilic properties such as a terminal carboxylic acid group may be linked to a ligand or small molecule having nucleophilic properties such as an amino group by means of condensation. Small molecules may be coupled to reasonably large ligands (up to 5000 D) to form hybrid ligands without significantly losing membrane permeability.

According to the method of the present invention, the hybrid ligand is introduced into a sample containing an environment as described above. The environment is characterized by a functional transcription and translation apparatus, and may be composed of whole cells, cell lysate or a synthetic mixture of enzymes and reagents. It is desirable that components of the assay including vectors and hybrid molecules be readily introduced into the environment. An example of an environment which is cellular would be an environment of eukaryotic cells, more particularly a yeast cell population, such as Saccharomyces cerevisiae or Schizosaccharomyces pombe; other examples would include invertebrate cell lines such as Drosophila cells or mammalian cells. Cells that are capable of use in the chemical-hybrid assay according to the present invention include those in primary cultures, cultures of immortalized cells or cells that have been genetically manipulated. Different cell types may be selected for the chemical-hybrid assay according to the present invention according to the permeability of the cells to selected hybrid ligands. Another criteria for selection of a particular cell type may be the nature of post translational modification of proteins expressed by the recombinant vectors where the binding of such modified proteins to a small molecule may more accurately mimic the natural state. The assay may be performed using single cells or populations of cells for each test sample.

According to the method of the present invention, the introduction of the hybrid ligand into the environment, may include traversing a membrane so as to enter the cell. In this situation, the hybrid molecule is introduced into cells by, for example, electroporation or other permeation procedures that are well known in the art. In certain embodiments, cells may be used that may be genetically or pharmacologically modified to increase the intracellular concentrations of the hybrid ligand. These include procedures that utilize polybasic peptides such as polymixin B or genetically altered strains of cells that offer increased permeability or decrease efflux of hybrid ligand. In addition, the appropriate hybrid ligand may be selectively formed having an overall charge and polarity that facilitates transmembrane transport.

According to an over-all depiction of a chemical hybrid assay conducted according to the present invention, the environment will contains three different types of vector. Two of the vectors encode fusion or hybrid proteins, each hybrid protein including a transcription module and a target molecule for binding ligand A or ligand B of the hybrid ligand. Once the chemical-hybrid complex is formed, and the transcription modules are brought into close proximity, the transcriptional activation of a reporter gene will occur as exemplified in Example 1.

Transcription factors bind to specific DNA sequences adjacent to the gene to be transcribed thereby facilitating the functioning of the transcriptional machinery. It is well established that many transcription factors possess two modular domains that are separable in function [see Curr Opin Biotechnol., 5(5):482 (1994)]. In eukaryotic transcription systems, the DNA binding module is not physically on the same peptide as the transcription activation module. The first module is responsible for recognizing the sequence specific DNA adjacent to a particular gene in the promoter region and the second is a more general module which consists of a number of acidic amino acid residues that act as transcriptional enhancers. Where the modules are encoded on separate vectors, an event is required that brings the transcription activating modules together so as to initiate transcription of the reporter gene.

Several transcriptional activation modules have been identified [see, for example, WO95/02684]. Any of these may be suited for use in the chemical-hybrid system. In particular, Example 1 utilizes the E. coli LexA DNA binding protein that binds tightly to LexA operator and activates transcription of a reporter gene such as Lac Z. A wide variety of transcriptional activation domains can be used including the bacterial B42 transcriptional activator GAL 4, (Example 2), GCN4 and VPI6. The DNA encoding transcriptional activator modules are incorporated into vectors that are capable of being expressed in eukaryotic cells. Adjacent to these sequences is inserted DNA encoding target protein (first expression vector) or unknown gene products (second expression vector) such that a fusion protein is expressed by the eukaryotic cell. Vectors containing transcription modules are described in the art and any of these may be used according to the assay described herein {see, for example, PNAS 93:12817, (1996)].

An application of the chemical hybrid assay is when the small molecule has a known pharmacological function but unknown target, the unknown targets being established by means of the assay (Example 1). The target molecule may be any cellular component including a nucleic acid, a polysaccharide, a lipid or a protein or a combination of any of these. In the examples provided below, the target is a protein encoded by DNA. Cloned DNA encoding target protein may be inserted by standard cloning techniques. Alternatively, random DNA sequences of a size that is capable of encoding a yet undetermined target protein, may be inserted in the second expression vector where the random DNA sequences are derived from a genomic DNA library, cDNA library or synthetically generated library formed from eukaryotic cells, prokaryotic cells, viruses or formed by an automated DNA synthesizer. Examples of target proteins encoded by a plasmid library may include enzymes, oncogene products, signaling proteins, transcription factors and soluble domains of membrane proteins. An alternative application of the chemical-hybrid assay is when the nature of the target molecule is known and a small molecule is sought that is capable of binding the target molecule. This type of assay may be a direct assay (Examples 1, 2) or a competitive binding assay (Example 5).

The third vector contained in the environment is a vector encoding a reporter protein which is switched on in the presence of united transcription activation modules. Reporter genes are so named because when transcribed and translated, they can be detected according to a phenotype based on a selectable characteristic such as growth in an appropriate growth media or visual screening. In a preferred embodiment of the invention, reporter genes that permit visual screening are utilized. Examples of reporter gene products that may be detected visually include β-galactosidase and Aequorea Victoria Green Fluorescent Protein (GFP), antibodies or selected antigens. These gene-products may be identified visually or by spectrophotometric quantification.

The switching on or off of the reporter gene depends in part on the binding affinity of the small molecule ligand to the target so as to activate the reporter gene or to competitively inhibit the activation of the reporter gene. The affinity of a ligand or small molecule for a target molecule may vary substantially in the chemical-hybrid screen. An example of a range of binding affinities includes a Kd having a value below 10⁻⁶, more preferably below 10⁻⁷ and even more preferably below 10⁻⁸ and in some embodiments below about 10⁻⁹. An example of a dissociation constant includes a range less than 10 mM. This does not preclude the effectiveness of a binding affinity outside this range. Ligand A may be selected on the basis of substantially defined structure activity data concerning binding to a known target; established chemistry for linking the ligand to a small molecule; and strong binding affinity for a target encoded by a fusion gene.

A feature of the chemical-hybrid system includes the formation of a hybrid ligand molecule. The consequence of the hybrid molecule binding to both target hybrid molecules is a chemical-hybrid complex that results in the stimulation of transcription of at least one reporter gene. The detection of a positive result may follow from direct binding of a hybrid ligand to target hybrid molecules or by competitive binding of the hybrid ligand acting as an agonist or antagonist. In certain circumstances, the target molecule for therapeutic intervention may be known but a suitable small molecule for binding the target molecule may be desired. If no candidate small molecule for binding the target is known, it may be desirable to generate a random library of hybrid molecules in which a mixture of small molecules are chemically modified in such a way as to bind to a preselected ligand. Subsequently, pools of molecular hybrids may be introduced into an environment such as yeast cells for performing the chemical-hybrid system. Those samples that are positive can be reanalyzed using increasingly smaller subsets of the initial pool until a single candidate small molecule type is discovered.

Alternatively, a candidate small molecule that binds a selected target molecule may be known, but it is desirable to select a small molecule with improved binding affinity for the target molecule. In this situation, a molecular hybrid of the candidate small molecule and a ligand is formed and the chemical-hybrid screening assay is performed in the presence of a library of small molecules that compete with the molecular hybrid for binding the target. Those samples which contain small molecules having improved binding to the target molecule, compared with the candidate small molecule, will not activate the reporter gene.

In one embodiment of the invention, a kit is provided containing a ligand with a suitably charged reactive group. The kit further includes reagents for attaching the ligand to a small molecule for utilization in a chemical-hybrid system. In another embodiment of the invention, a kit is provided for practicing the method of the invention. The kit may include a reaction chamber, at least two vectors, a host cell and a ligand with a suitably charged reactive group for reaction with a small molecule. The two vectors encode hybrid proteins as described below in Example 1.

### EXAMPLE 1

### Compounds Which Irreversibly (Covalently) Bind to Targets: Synthesis of Hybrid Molecules

### Example 1a

### Photoaffinity Labeling Agent Analogs

(i) Modified Aspirin Analogs

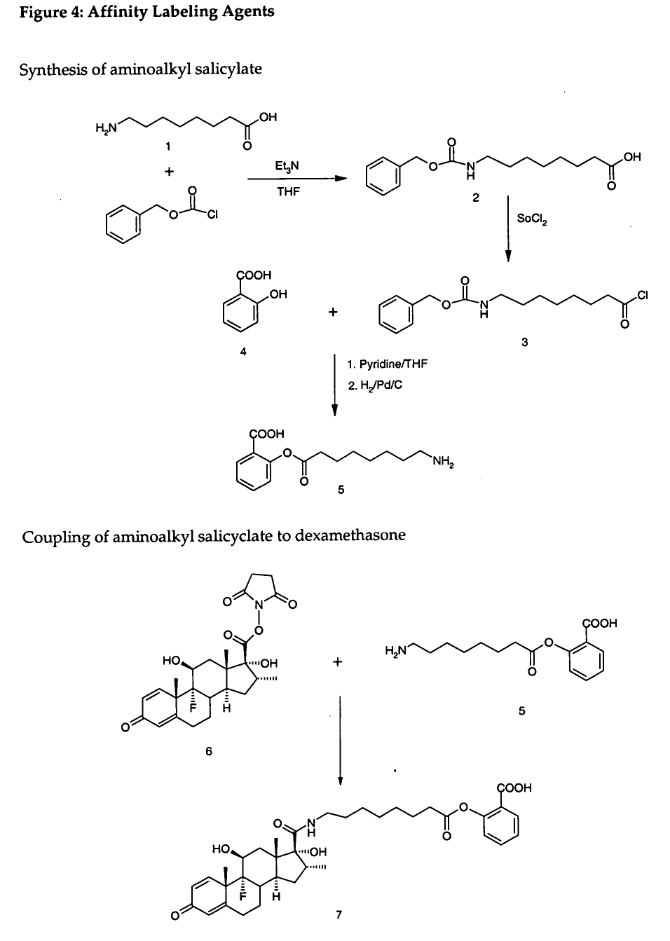

Modified analogs of aspirin (hybrid molecule) were prepared using salicylic acid as a starting material. The intermediate aminoalkylsalicyclate derivatives (4) were prepared starting from an aminoalkylacid (1). The amino group was protected to yield 2 followed by formation of the acid chloride (3) using standard methods. The salicyclic acid reaction with acid chloride followed by deprotection resulted in 5. The aminoalkylsalicyclate (5) was coupled to the dexamethasone acid derivative (6) by N-hydroxysuccinimide activation to yield dexamethasone coupled aminoalkylsalicyclate (7) as shown in reaction schemes in FIG. 4. Similarly FK-506 can be coupled with aminoalkylsalicyclate to produce the hybrid molecule. The mixed carbonate of FK506 can be prepared by the literature method (Pruschy, (1994) Chem. Biol. 1,163-172) and dexamethasone alkylamine (Licitra, PNAS 93,12817,1996) can be prepared from dexamethasone which can subsequently be coupled to aminoalkyl salicylate.

(ii) Modified β-Lactam Analogs

Hybrid molecules using the antibiotics for irreversible bonding to the target are synthesized by replacing the functional groups R and/or Y with the another small molecule as shown in FIG. 5. The □-lactams form an covalent bond with the transpeptidases via acylation of the serine hydroxy group. The structure activity relationship of β-lactams to form irreversible (covalent) bond with the target protein can be exploited by anybody skilled in the art to create novel set of hybrid molecules. The synthesis of the exemplary compounds listed in Tables 1 and 1 A is well established and the desirable small molecules (e.g., examethasone and FK-506) can be introduced during the synthesis by standard synthetic procedures.

TABLE 1
List of irreversible enzyme inhibitors (Affinity labeling agents)

| Drugs in | | Affinity Labeling | |
|---|---|---|---|
| Clinic: | Disease/Indication | Agents | Enzyme Targets |
| 1 | NSAIDS | Aspirin | Cyclooxygenase |
| | Antibiotics: | | |
| | Penicillins (6) | | |
| 2 | | Penicillin G | Transpeptidase |
| 3 | | Penicillin G | Transpeptidase |
| 4 | | Oxacillin | Transpeptidase |
| 5 | | Cloxacillin | Transpeptidase |
| 6 | | Ampicillin | Transpeptidase |
| 7 | Cephalosporins/ | Amoxicillin | Transpeptidase |
| | Cephamycins (4) | | |
| 8 | | Cefazolin | Transpeptidase |
| 9 | | Cefoxitin | Transpeptidase |
| 10 | | Cefactor | Transpeptidase |
| 11 | | Ceftizoxime | Transpeptidase |

TABLE 1A

| Penicillins | Cephalosporins/Cephamycins |
|---|---|
| Cloxacillin | Cefazolin |
| Penicillin G | Cefoxitin |
| Oxacillin | Cefaclor |
| Cloxacillin | Ceftizoxime |
| Ampicillin | |
| Amoxicillin | |

TABLE 2
List of irreversible enzyme inhibitors (Suicide inhibitors):

| | |
|---|---|
| Drugs/Irreversible | |
| Inhibitors - | Enzyme Targets |

Compounds
in Clinic:

| | |
|---|---|
| α-Diflouromethylomithine | Omithine decarboxylase |
| Vigabatrin | GABA aminotransferase |
| Allopurinol | Xanthine Oxidase |
| Tranylcypromine | Monoamine Oxidase |
| Pheneizine | Monoamine Oxidase |
| Hydralazine | Monoamine Oxidase |
| Pargyline | Monoamine Oxidase |
| L-Deprenyl | Monoamine Oxidase |
| Selegiline | Monoamine Oxidase |
| Clavulanic | β-Lactamases |
| Sulbactam | β-Lactamases |
| 5-Fluoro-2-deoxyuridylate | Thymidylate Synthase |
| Trifluridine | Thymidylate Synthase |
| Methimazole | Thyroid Peroxidase |
| Methylthiouracil | Thyroid Peroxidase |
| Propylthiouracil | Thyroid Peroxidase |
| Chloramphenicol | Cytochrome P-450 |
| Norethindrone | Cytochrome P-450 |
| Halothane | Cytochrome P-450 |
| Fluoroxene | Cytochrome P-450 |
| Ethclorvyol | Cytochrome P-450 |
| Spironolactone | Cytochrome P-450 |
| Danazol | Cytochrome P-450 |
| Methhoxsalen | Cytochrome P-450 |
| Novonal | Cytochrome P-450 |
| Compounds Listed | |
| in Literature: | |
| MedLine Search | |
| a-Ketoheterocyclic | Human Neutrophil Elastase |
| Acetylenic indolalkylamine | Monoamine Oxidase |
| 6-(Bromoethylene) Pyran-2-one | Phospholipase A2 |
| 7-Substituted Androstatriene | Aromatase |
| 3-Amino-1,2,4-triazole (Amitrole) | Lactoperoxidase |
| Lophotoxin | Nicotinic Receptors |
| Tamoxifin Aziridine | Estrogen Receptors |
| 2-Thioadenosine | Epidermal Growth Factor Receptor |
| 2-Alkyl insonic acid | Inosine monophosphate |
| | dehydrogenase |
| Leukotriene A4 hydrolase | Protein (1 stoichiometry) |
| 4-(Fluoromethyl) phenyl phosphate | Calcineurin |
| Octapeptide | Protein Kinase C |
| Haloperidol derivatives (10) | HIV Proteases |
| Clorgyline Analogues | Monoamine Oxidase |
| O-(epoxyalkyl) tyrosine | Serine Proteases |
| Phenylpropynal | β-Lactamases |

### Example 1b

### Modified Mechanism-Based Inactivator Analogs

The mechanism based inactivators have natural tendency to form covalent bond with their targets. The initial step is to form an reversible complex with the target during which reactive chemical species is generated to form covalent bond with the target. The examples of such compounds are listed in Table 2 and the synthetic strategies are showed in FIGS. 6a-6c for three prominent drugs such as vigabatrin, eflornithine, and fluorouracil.

### Example 1c

### Modified Fluorescein Analogs

Recently specific covalent labeling of recombinant protein molecules in living cells has been reported using the fluorescein-EDT2 analog (Science, 281, 269 (1998)) as shown in FIG. 7. Fluorescein has free carboxylic acid group on one of the aromatic ring. This carboxylic acid is used for coupling to other small molecules. As an example dexamethasone alkylamine is coupled to fluorescein carboxylic acid group using standard coupling reaction conditions outlined in FIG. 7.

### Example 1d

### Enzyme-Catalysed Covalent Coupling of Proteins/Peptides With Small Molecules

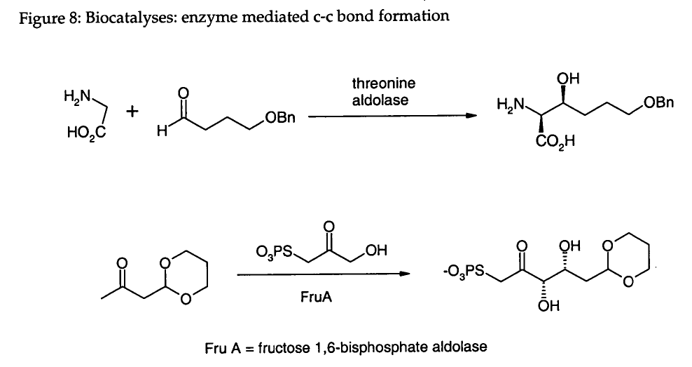

The use of an enzyme for organic coupling reactions is a well known. As an example C—C bond formation between an aidehyde and CH₂ group by an enzyme threonine aldolase is easily accomplished (FIG. 8). The method in the invention will exploit coupling of a small molecule (ligand A) to the protein or a peptide inside the cell. It will involve expression of the specific target protein and/or peptide in the yeast cells (or a recombinant (mutant) libraries of proteins and peptides. The target library of protein and/or peptide is created with a bias to have desired functionality (e.g., NH2, COOH, OH, SH) for efficient covalent bonding (to modulate the rate of reaction) by an enzyme. The enzyme may be an inducible. The small molecule may have a linker to keep the small molecule apart from the target protein/peptide. Anybody skilled in the art can envision and design the system in the invention.

### Example 1e

### Combinatorial Biocatalyses

Hybrid molecules can be synthesized by natural processes reported in the literature. Iterative reactions can be performed using isolated enzymes or whole cells, in natural and unnatural environments, and on substrates in solution or on a solid phase. Always one of the ligands is selected which has affinity for a predetermined target (forms a covalent bond) and the other ligand is compounds generated by combinatorial biocatalyses and are coupled under the reaction conditions described here (Trends Biotechnol., 1998 May; 16 (5): 210-215).

### Example 1f

### Construction of Vectors Encoding Target Proteins

The vectors encoding the targets for ligand A (and Ligand B) in the hybrid molecule as summarized in Table 3 were cloned into binding or activation domain plasmid vectors by standard recombinant DNA protocols or by the gap-repair protocols. Both wild-type and mutant proteins and relevant protein domains are produced for testing in chemical-hybrid system. In addition, to biological proteins (natural) or protein domains recombinant proteins will be engineered to selectively and irreversibly bind the ligand A.

One example of the invention utilizing yeast strains containing the LexA operator, and LacZ and Leu 2 reporter genes is described in detail below. A second example utilizing another yeast strains containing Gal 4 operator and Lac Z and Ura 3 reporter genes is also described. Other operator/reporter gene combinations although not described are suitable for use in the chemical-hybrid assay.

TABLE 3

| Ligands A (or B) | Targets |
|---|---|
| Aspirin | Cyclooxygenase |
| B-lactams | Peptidoglycan |
| Fluorescein-EDT2 | Recombinant peptide |
| FK506 | FKBP12 |
| Vigabatrin | GABA aminotransferase |
| Eflornithine | Ornithine decarboxylase |

Specific examples for cloning of exemplary targets are described below:

Construction of a Vector Encoding a Hybrid Protein of FKBP (or Rat Glucocorticoid Receptor)-Transcriptional Activator

A first vector containing the cDNA fragment encoding FKBP12 (or rat glucocorticoid receptor) transcriptional module are formed as follows. The cDNA encoding FKBP 12 was originally obtained from a human cDNA library prepared according to well known techniques (Current Protocols in Molecular Biology). The cDNA encoding the FKBP12 was amplified by PCR and subcloned into the EcoRI and XhoI sites of the pJG4-5 vector where the pJG4-5 vector already contains the transcriptional activator module. (Current Protocols in Molecular Biology). The resulting vector is called pJGFKBP.

Construction of the Vector Encoding the Hybrid Protein of Appropriate Receptor-LexA DNA Binding Domain.

A second vector encoding the appropriate target receptors (e.g., cyclooxygenase, transpeptidase, recombinant protein/peptides) and the LexA binding protein are made as follows: A clone containing the appropriate target receptors is obtained according to standard protocols. A fragment encoding amino acid residues of the protein with the was generated by a standard PCR reaction. The fragment is flanked by the appropriate restriction sites and is subcloned into the EcoRI and XhoI sites of the pEG202 vector (Current Protocols in Molecular Biology) where the pEG202 vector contains the sequence which encodes for a protein which binds the bacterial LexA operator. The resulting hybrid construct, encodes the second hybrid protein in the assay.

A third vector identified as pSH18-34 and containing the lacZ reporter gene downstream of a number of LexA operators was made following standard techniques.

Yeast Strain

Saccharomyces cerevisiae (EGY 48), was transformed with the three vectors described above using standard lithium acetate transformation procedures. Positive transformants were selected by plating cells on complete minimal media yeast dropout plates containing 2% glucose, and lacking histidine, tryptophan and uracil. The transformed EGY48 yeast were then screened as described below.

Chemical Hybrid Screen and Appropriate Controls

The appropriate hybrid ligand (from examples 1a to 1e), will be introduced into a population of yeast cells in two different experiments which had previously been transformed with vectors encoding: the LexA DNA Binding Domain corresponding receptors for the hybrid ligand: lacZ reporter; and transcriptional activator-FKBP12. The transformed EGY48 strain is plated onto complete minimal media Ura-,His-, Trp- yeast dropout plates containing 2% galactose, X-Gal, and hybrid ligand. A light blue color will signifying reporter gene activation, This experiment will demonstrate that the complex could be formed in vivo This experiment can also be performed on similar plates which were also leu-. The leu 2 gene is used in EGY48 as a second reporter gene. Only yeast will grow in the absence of leucine containing a complex.

A competitive assay can also be performed as an additional control. The above yeast strain is plated onto complete minimal media Ura-, His-, Trp- yeast dropout plates containing 2% galactose, hybrid ligand and competing ligand (dexamethasone and FK506). If all the yeast remained white in this will confirm that competing ligand (dexamethasone and FK506) competitively inhibited the formation of the complex required for activation of the Lac Z gene, and underscored the specificity of the ligands for the target molecules.

Isolation of cDNA Clones Expressing Protein That Binds Hybrid Ligand

The yeast strain: EGY48 ura3 trp 1 his3 LexA operator-LEU 2; will be transformed with appropriate vectors and plated onto synthetic complete (SC) medium (His-, Ura-). The resultant EGY48 harboring vectors is transformed with a Jurkat cDNA library subcloned into pJG4-5. The transformed yeast cells (1.62×106) are plated onto SC medium (pH 6.5, His-, Ura-, Trp- Leu-) containing galactose and hybrid ligand. Colonies will be collected and plated onto SC medium (His-, Ura-, Trp-, Leu-) containing galactose. Colonies that displayed growth independent of the presence of hybrid ligand will be discarded. The remaining colonies will be plated onto SC medium (pH 6.5, His-, Ura-, Trp-, Leu-) containing galactose and hybrid ligand in the presence of competing ligand (dexamethasone and FK506). Those colonies whose growth could be completely inhibited by the competing ligand will be grown in liquid culture. The hybrid vectors containing cDNA fused with a transcription activation module will be retrieved from yeast strains and transformed into E. cold DH5a for preparation of the plasmids. The DNA inserts in these plasmids will be sequenced by an ABI automated sequencer and to identify the encoding protein.

### EXAMPLE 2

### Identification of the Cellular Component That Binds to Dexamethasone (and FK506) Using a Yeast System Based on Gal4 DNA-Binding Domain and Activation Domain

A chemical-hybrid assay using a second Gal4 DNA binding domain and activation domain as described in U.S. Pat. No. 5,468,61 was also tested. The appropriate target receptor containing either a no mutations or mutations are PCR amplified using primers tagged with restriction sites and subcloned into the vector pASII to encode a fusion protein between the Gal4 DNA binding domain and appropriate target receptor to give a plasmid. The coding sequence of rat glucocorticoid (and human FKBP12) are PCR amplified and subcloned into the vector pACTII to include a fusion protein between Gal4 activation domain and rat glucocorticoid receptor (and human FKBP12). The resultant vectors are transformed into the yeast strain Y190 using lithium acetate method and the transformed yeast were selected on SC (Leu-, Trp-). The transformed yeast strain, are streaked on plates (Leu-, Trp-, His-) containing 30 mM 3-aminotriazole and 1 mM hybrid ligand in the presence or absence of a competing ligand (dexamethasone or FK506). It is expected that on plates that lack competing ligand (dexamethasone or FK506) colonies will grow but will be absent from the plate containing the competing ligand. These experiments will confirm that the chemical-hybrid interaction can be established in the yeast system based on Gal4 DNA-binding domain and activation domain. Furthermore, these experiments will demonstrate that this yeast system can be used for screening for ligands that compete for an established chemical-hybrid ligand protein interaction. This yeast system has both a His- biosynthetic gene and a LacZ reporter gene as reporters for detection of chemical-hybrid interactions to allow galactosidase assay.

### EXAMPLE 3

### Identification of a Small Molecule Capable of Binding to a Selected Target Molecule

A population of yeast cells which have previously been transformed with vectors according to Example 1 where the first hybrid protein any of the target receptor (e.g., cyclooxygenase, transpeptidase) fused to LexA DNA-binding domain, and the second hybrid protein is rat glucocorticoid receptor (or FKBP12) fused to a transcriptional activator module and the reporter gene is Lac Z (and Ura3). A 96-well plate is prepared such that each well contains a single member of the hybrid ligand library composed of ligand A covalently (e.g., aspirin, □-lactams, vigabatrin, and fluorescein) linked to a library of small molecules. The transformed yeast is grown in each well and a blue coloration is looked for (growth of colonies with Ura3). Those wells expressing the reporter gene are identified and structural information on the corresponding hybrid ligand is retrieved.

### EXAMPLE 4

### Competitive Assay for Identifying a Small Molecule Ligand Having a Binding Affinity for a Known Target

A population of yeast cells which have previously been transformed with vectors according to Example 1 are placed in a 96 well dish. These yeast cells were transformed with DNA encoding a first hybrid protein which is the target receptor (e.g., cyclooxygenase, transpeptidase) fused to LexA DNA-binding domain, and a second hybrid protein which is glucocorticoid receptor (and FKBP12) fused to a transcriptional activator module and a third vector containing the reporter Lac Z gene (and Ura3). A single member of a ligand library covalently linked to a hybrid ligand prepared according to Example 1 was added to each well containing the yeast. Those wells which were identified as having a blue coloring were scored as negative while those wells that appeared white were scored positive. Control wells having either hybrid ligand only or no hybrid molecule were included. The samples are identified according to the absence of expression of the reporter gene; and the ligand from the library is characterized so as to determine its structure information.

### EXAMPLE 5

### Assay for Identifying a Diagnostic Reagent for Screening for Small Molecule Contaminants in the Environment

A cDNA transcriptional activator fusion library is prepared from immune cells (B-cells) capable of producing antibodies to a specific small molecule contaminant, in this case, DDT. Using the screening assay described in Example 1, a hybrid molecule is formed from irreversible ligand A/DDT. Yeast cells are transformed accordingly with the cDNA fusion library, a vector encoding the hybrid protein containing binding domain of the target receptor and a vector encoding the reporter gene Lac Z (and Ura3) and the hybrid ligand is introduced so as to identify target molecules. The positive clones are identified by the blue coloration (and growth of colonies). The vector containing the cDNA from positively staining cells is isolated and the protein product utilized as a reagent in environmental screening assays to detect DDT with high affinity.

### EXAMPLE 6

### A Chemical Hybrid Screening Kit

A kit is prepared that contains a plasmid encoding the LexA DNA binding module fused to the target receptor according to Example 1; a plasmid encoding the transcriptional activation domain fused to fragments in a cDNA library: and a reporter plasmid containing Lac Z, Ura3, GFP or luciferase. The cDNA library for use in the kit is selected from a variety of sources including T-cells, cardiac cells and liver cells. the choice being dependent on the characteristics of the potential target protein and the small molecule. The kit contains a conserved ligand for reacting with a small molecule to form a hybrid molecule by standard coupling procedures described in FIGS. 3-8. Although a number of linkages may be exploited including ester, ether and amide bonds. In addition, the kit provides an environment. in this case, yeast cells, for permitting the chemical hybrid screening assay to occur.

Thus, while we have illustrated and described the preferred embodiment of our invention, it is to be understood that this invention is capable of variation and modification by those skilled in the art to which it pertains, and we therefore do not wish to be limited to the precise terms set forth, but desire to avail ourselves of such changes and alterations which may be made for adapting the invention to various usages and conditions. Accordingly, such changes and alterations are properly intended to be within the full range of equivalents, and therefore within the purview of the following claims.

Having thus described our invention and the manner and a process of making and using it in such full, clear, concise and exact terms so as to enable any person skilled in the art to which it pertains, or with which it is most nearly connected, to make and use the same;

1. A method for identifying a cellular component to which a small molecule is capable of binding, comprising:
(a) providing a hybrid ligand consisting essentially of two ligands linked together, wherein one ligand has specificity for a predetermined target and forms an irreversible (covalent) bond therewith; and a second ligand that is the small molecule:
(b) introducing the hybrid molecule into a sample containing an environment, the environment having;
(i) a first expression vector, including DNA encoding the target for said one ligand, linked to a coding sequence for a first transcriptional module for expression as a first hybrid protein;
(ii) a second expression vector including a random DNA fragment encoding a polypeptide linked to a second transcriptional module for expression as a second hybrid protein; and
(iii) a third vector including a reporter gene wherein the expression of the reporter gene is conditioned on the proximity of the first and second hybrid proteins;
(c) permitting the hybrid molecule to bind covalently the first hybrid protein through said one ligand, and the second hybrid protein through said second ligand so as to activate the expression of the reporter gene; and
(d) identifying those samples expressing the reporter gene,

2. A method according to claim 1 which further comprises
(e) characterizing the second hybrid protein in the samples identified in (d) so as to determine the cellular component to which the small molecule has a binding affinity. transcriptional activator.

3. A method according to claim 1 which further comprises
(e) characterizing said second ligand so as to identify the small molecule capable of binding the molecular target.
